(19) United States
(12) Patent Application Publication
(10) Pub. No.: US 2004/0076969 A1
(43) Pub. Date: Apr. 22, 2004

(54) Method for detecting known mutations in tube

(76) Inventors: Fabrice Cailloux, Clermont-Ferrand (FR); St?eacute;phane Gobron, Clermont-Ferrand (FR)

Correspondence Address: Foley & Lardner, Washington Harbour Suite 500, 3000 K Street N W, P O Box 25696, Washington, DC, 20007-8696, US

(21) Appl. No.: 10/344,093

(22) PCT Filed: Aug. 8, 2001
(86) PCT No.: PCT/FR01/02574

(30) Foreign Application Priority Data
Aug. 8, 2000 (FR) 0010425

(51) Int. Cl. C12Q 1/68

(52) U.S. Cl. 435/6

(57) ABSTRACT

The invention concerns a method for detecting a mutation in a target nucleic acid comprising amplification of the target DNA, specific hybridization of a probe with the DNA target, extension of the probe by selective addition of αS-phosphothioat-edesoxynucleotide complementary of the mutation in a tube A and adding an αS-phosphothioatedesoxynucleotide complementary of the natural base corresponding to said mutation in a tube B; the resulting extended probe being resistant to digestion by an exonuclease, particularly by exonuclease III.

The present invention relates to a method for detecting a mutation in a target nucleic acid, comprising amplification of the target DNA, specific hybridization of a probe with the target DNA, extension of the probe with selective addition of an αS-phosphothioate deoxynucleotide complementary for the mutation in tube A and addition of an αS-phosphothioate deoxynucleotide complementary for the natural base corresponding to said mutation in tube B, the probe thus extended being resistant to digestion by an exonuclease, in particular exonucleases III. The detection of the mutation and of the natural base is carried out by direct or indirect measurement in tubes A and B.

Mutations in germinal cells or in somatic lines can have dreadful consequences on the organism by causing, for example, hereditary genetic diseases or the appearance of cancer. The effect of a mutation mostly depends on its location in the DNA. In the case of a mutation in a coding region, loss of the function of the encoded protein may be observed. If the mutation is in a regulatory region, the expression of the DNA may be destroyed or increased. A mutation in a gene involved in cancer in the germinal line does not necessarily mean that the individual concerned will effectively contract a tumor, but only that the risk thereof is increased. In addition, when seeking to diagnose the invasive potential of an already established tumor, it is not known in advance which mutations should be expected since they may be on several genes or at several sites of the same gene. Consequently, it appears to be necessary to have a simple, rapid and reliable test which can readily detect many mutations.

The need for a technique for detecting mutations, for typing or else for studying polymorphism is being increasingly felt in industry, either to allow the discovery of novel biological targets of interest, or to determine precisely the genetic profile of a tumor or of a patient and to envision suitable treatments. This need has led to the development of various techniques, such as LCR, SSCP and RFLP, but they remain difficult to implement on a large scale.

The aim which is the basis of the present invention has been to develop a technique for the rapid and easy determination of nucleotides to be identified and, consequently, the diagnosis of mutations and of poly-morphisms of genes, or the identification of pathogenic or genetically modified microorganisms. More specifically, the problem lies in a compilation of various techniques combining, within the same tube, amplification of the DNA of a sample and detection of a given nucleotide. This method proves to be easy to use, and generates signals which do not require tedious processing and complicated interpretation.

In the context of the present invention, a system has been developed in which the set of steps leading to the results is carried out in the same tube, and which is based on amplification of the target DNA, specific hybridization of a probe (which in the present case serves as an oligonucleotide primer) with the target DNA, and extension of the probe with selective addition of an αS-phosphothioate deoxynucleotide at the 3′ end of the primer complementary to the target DNA, the primer thus extended being resistant to digestion by an exonuclease, in particular by exonuclease III. Thus, the probe remains present in the tube only when the following conditions come together:

a) hybridization between the probe and target DNA of the sample, and

b) presence of a complementary base in the target DNA allowing incorporation of the αS-phosphothioate deoxynucleotide into the probe; which prevent its degradation by the nuclease.

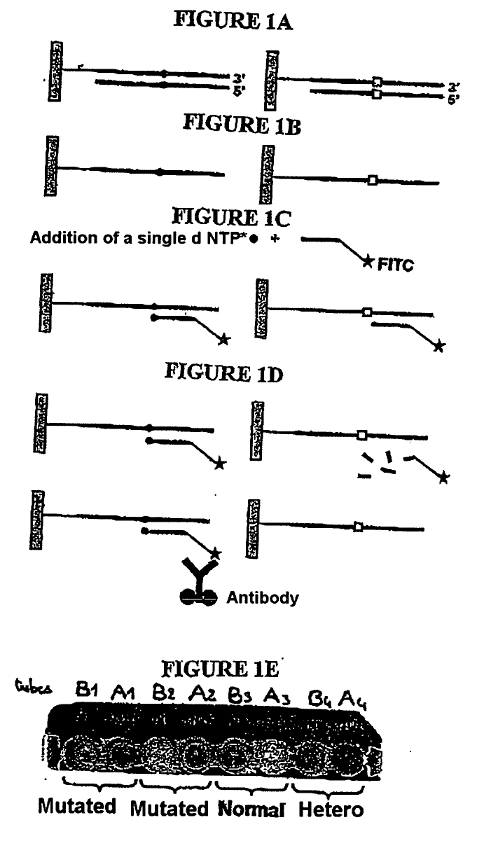

The key steps of this method are illustrated in the example given in FIG. 1 hereinafter.

The technique described in U.S. Pat. No. 4,656,127 consists of the incorporation of a thio-dNTP which protects against exonuclease degradation. However, the method according to the invention offers the advantage of rapidity, simplicity of use and ease of interpretation of the results. Specifically, the use of two different tubes A and B, one being intended for detection of the mutated base (tube A) and the other being intended for detection of the normal base corresponding to said mutated base (tube B), allows excellent reliability (presence of a positive control, whatever the genotype to be tested) and direct interpretation of the results. A greater number of tubes can also be used. Thus, in a particular and advantageous case, four tubes are used, in order to be able to test the presence of each base on the target DNA. This makes it possible to refine the analysis when the base tested is very polymorphic.

A second advantage of the method according to the invention is imparted by the attachment of one of the primers to the tube used for the amplification and thus to anchor the amplification products to the walls of the tubes. The advantage of this anchoring is to be able to subsequently carry out all the reactions leading to the colored revelation, including washes, in the same tube, which in particular avoids risks of contamination and represents a considerable gain in time, especially given that using the same solid support for all the steps of the method facilitates automation.

A third advantage of the present invention is to differentiate homozygous and heterozygous carriers of the targeted mutation, through using two different tubes. Specifically, the revelation results in a coloration in just one of the two tubes if the mutation is present homozygously, whereas the coloration is present in both tubes if the mutation is present in the heterozygous state.

Moreover, the result can be obtained in 30 to 40 minutes after PCR amplification and does not require any particular tiresome environment. In addition, some steps can be combined (exonuclease and revelation in particular) in order to decrease the time for implementation without however, modifying the specificity.

By virtue of such a method, the detection experiment can be repeated, unlike the technique described in U.S. Pat. No. 4,656,127. Specifically, use of the solid support makes it possible to conserve the strand of nucleic acid to be analyzed and the exonuclease degrading the unprotected probe can be replenished and the hybridization step and detection step can be repeated in the same tube if need be.

The industrialization of such a method thus appears to be much easier since carrying out all the steps in the same tube, including the PCR, the various incubations and the various washes, make it possible to envision rapid automation of the method on any marketed automaton platform for molecular biology.

### DESCRIPTION

The present invention relates to a method for identifying a known mutation or polymorphism in a nucleic acid sequence, comprising amplification of the region of interest by PCR, this amplification step being advantageously carried out using a primer attached to the wall of two tubes A and B (asymmetric PCR); hybridization of a specific probe designed in such a way that its end is positioned just upstream of the base to be revealed; extension of this probe by means of a polymerase with a modified nucleotide, advantageously a phosphothionucleotide, in particular an αS-phosphothioate deoxynucleotide, complementary to the mutation to be revealed (tube A) and complementary to the normal base corresponding to the mutation to be revealed (tube B); action of an exonuclease such that only the extended probes are not degraded. Detection of the presence or of the absence of a mutation is carried out by direct or indirect revelation of the probe. Advantageously, all the steps of the method are carried out in the same tube (from the PCR amplification to the revelation) such that the presence of a mutation is revealed directly by the appearance of a coloration in the tube which was used for the PCR (see FIG. 1). The presence of the mutation can also be detected by other detection methods, in particular fluorescence, or using radioactive probes (see below).

Thus, in a first aspect, the invention relates to a method for detecting a mutation occurring at position n in a target nucleic acid, characterized in that it comprises the following steps:

a) amplification on at least two different solid supports A and B of the region of interest comprising said mutation using at least one primer bound in the 5′ position to the supports,

b) dehybridization of the DNA strands and removal of the strands of the suspension by washing,

c) hybridization of a probe with the DNA strands bound to the solid supports A and B, the 3′ end of said probe hybridizing up to at most nucleotide n-1 of said strands,

d) reaction consisting of elongation of the probe hybridized in step c), by incorporation, in the 5′-3′ direction, of nucleotides complementary to said DNA strands using a reaction mixture comprising a DNA polymerase and a nucleotide derivative resistant to degradation by an exonuclease (dNTP*), the reaction mixture used for the support A comprising a DNTP* complementary to said mutation and the reaction mixture used for the support B comprising a dNTP* complementary to the normal base corresponding to said mutation,

e) degradation with said exonuclease such that only the probes elongated in step d) are not degraded, washing,

f) direct or indirect revelation of the nondegraded probe, said revelation being positive either when the DNA strand on the support A contains the mutation, or when the DNA strand on the support B contains the normal base corresponding to said mutation.

The binding of the primer, in the 5′ position, to the supports (step a) is advantageously binding which is sufficiently strong for the DNA to remain attached to the support during the various steps of the method. This may be a covalent bond, but also noncovalent binding, such as avidin/streptavidin/biotin binding, or binding of the antibody/antigen type.

In step c), the expression “the probe hybridizing up to at most nucleotide n-1 of said strands” means that the probe can also hybridize at n-2, n-3, n-4, etc. It will then therefore be necessary to provide, in the elongation step (d), the nucleotides complementary to the missing bases in order to enable the elongation of the probe up to the mutated base for which the dNTP* is provided.

A “probe” is defined as being a nucleotide fragment comprising, for example, from 10 to 100 nucleotides, in particular from 15 to 35 nucleotides, having a specificity of hybridization under given conditions so as to form a hybridization complex with the target nucleic acid. The probes according to the invention may carry a labeling agent allowing or enhancing their detection.

Of course, the probe is also used as a primer in the context of the invention, since the aim is to incorporate a modified nucleotide at position n corresponding to the position of the mutation being sought. The 3′ end of the probe therefore ends at most, and preferably, at n-1.

The probe may be labeled using a label chosen, for example, from radioactive isotopes, enzymes, in particular enzymes capable of acting on a chromogenic, fluorigenic or luminescent substrate (in particular a peroxidase or an alkaline phosphatase) or else enzymes which produce or use protons (oxidase or hydrolase), chemical chromophore compounds, chromogenic, fluorigenic or luminescent compounds, nucleotide based analogs, and ligands such as biotin. The labeling of the probes according to the invention is carried out with elements selected from ligands, such as biotin, avidin, streptavidin or dioxigenin, haptens, dyes, and luminescent agents such as radioluminescent, chemiluminescent, bioluminescent, fluorescent or phosphorescent agents. Another possibility is to label the probe with a peptide comprising an epitope recognized by a given antibody. The presence of this antibody can be revealed using a labeled second antibody.

In this method, the probe is advantageously labeled with one of the abovementioned molecules, said molecules allowing the direct or indirect appearance of a coloration when the nondegraded probe is present on the support, said coloration possibly being detected preferably by optical reading or simple observation. The term “optical reading” is intended to mean any measurement of absorption, of transmission or of emission of light which may possibly be at a wavelength specific either directly for the DNA (260 nm for example), or for any labeling molecule bound to the probe. This definition also comprises any measurement of the fluorescence emitted by labels (fluorescein and/or phycoerythrin).

Advantageously, the presence or absence of the mutation is therefore detected, in step f), either by optical reading, or by simple observation of a coloration on the solid support.

The solid support used to anchor the primer can be a plastic material (polystyrene, polycarbonate for example), or else nylon or glass, such that this support is a tube, a membrane or a bead on which the region of interest is amplified in order for it to be analyzed, i.e. detection of a base change.

Advantageously, the support is a tube (individual or in strips or in plates) used for the amplification, and therefore compatible with the various thermocyclers (Perkin Elmer 9700 for example). The tubes may also be any tubes or plate of the ELISA type, to which covalent attachment of nucleic acids may be carried out.

Various methods for immobilizing an oligonucleotide on a support are possible and are in particular described in U.S. Pat. No. 6,030,782. These methods may be direct immobilization by chemical bonding for example, or indirect immobilization (via a linker or a conjugate such as streptavidin for example).

In a preferred embodiment, the supports A and B are tubes to which covalent attachment of nucleic acids can be carried out. Preferably, these tubes are Nucleolink™ strips (NUNC, catalog reference No. 248259) which have a specific coating allowing the direct covalent attachment of oligonucleotides via their 5′ phosphate ends (S. R. Rasmussen, et al., Anal. Biochem, 198:138-142 (1991)). The format of this method is thus the standard 96-well format (12 strips of 8 wells), but since the strips can be broken up, the desired number of tubes can be used. Other formats can be used, in particular if other methods of attachment are used. For example, it is possible to attach the nucleic acids by a disulfide bridge according to the method recommended in U.S. Pat. No. 6,300,782.

In step d), an αS-phosphothioate deoxynucleotide is preferably used, preferably αS-dATP, αS-dTTP, αS-dCTP, αS-dGTP, αS-dUTP or αS-dITP, and in step e), an exonuclease is preferably used, in particular exonuclease III. The term “exonuclease” is intended to mean any natural or modified enzyme having exonuclease activity. It is also possible to envision the use of DNA polymerases having pyrophosphorolysis activity (in the presence of a high concentration of pyrophosphate, this enzyme adds a pyrophosphate to the last phosphodiester bond and therefore releases the nucleotide in the 3′ position). This product is available from Promega under the brand name READIT™, and variants using a system of revelation with luciferase is available under the brand name READase™.

In general, a well receives a DNA and then a probe, and the incorporation of just one nucleotide (dNTP*) is allowed. Therefore, at least two tubes are necessary for each DNA to be tested, one receiving the normal base and the other receiving the mutated base.

Thus, the method described above is characterized in that two tubes are used, one being intended for detection of the mutated base (tube A) and the other being intended for detection of the normal base corresponding to said mutated base (tube B).

Of course, use may be made of several series of tubes A and B, in particular in a 96-well plate, and a tube C as a negative control for each series of tubes A and B. In the preferred embodiment, said dNTP* is an αS-phosphothioate deoxynucleotide, such as αS-dATP, αS-dTTP, αS-dCTP, αS-dGTP, αS-dUTP or αS-dITP, which is incorporated at the 3′ end of the probe. This may take place, for example, by LCR, or preferably by asymmetric PCR. The αS-phosphothioate deoxynucleotides can be readily incorporated into polynucleotides using any of the polymerases and reverse transcriptases tested, which makes it possible to use DNA polymerases with a cost price more advantageous than in other mutation detections. The term “DNA polymerase” is intended to mean any natural or modified enzyme having polymerase activity. Mention may, for example, be made of the DNA pol exos-, in particular T7 or the klenow fragment.

Advantageously, steps e) and f) are carried out simultaneously in the same reaction mixture comprising said exonuclease and the means necessary to reveal the coloration. These means depend on the label or on the ligand which is on the probe.

Another aspect of the invention concerns a device or kit for carrying out the method defined above.

Such a kit is characterized in that it comprises:

tubes A and B in which at least one primer for amplifying the region comprising the specific mutation is bound, in the 5′ position, via a covalent bond,

reaction mixtures A and B, each comprising a different αS-phosphothioate deoxynucleotide selected from αS-dATP, αS-dTTP, αS-dCTP, αS-dGTP, αS-dUTP and αS-dITP, the reaction mixture used for the support A comprising an αS-phosphothioate deoxynucleotide complementary to said mutation and the reaction mixture used for the support B comprising an αS-phosphothioate deoxynucleotide complementary to the normal base corresponding to said mutation.

This kit may also comprise at least one element selected from an exonuclease, in particular exonuclease III, a reagent for revealing the coloration, a DNA polymerase, and various buffers or solutions required to carry out the method.

Advantageously, the kit according to the invention comprises a series of tubes A and B, each series making it possible to detect a given mutation, and tubes C as negative controls. Said tubes may be NUNC tubes.

In another aspect, the invention relates to the use of the method and of the kit described above, for detecting mutations of genes involved in diseases, in particular in hereditary genetic diseases, in particular hemochromatosis, sickle cell anemia, β-thalassemia and α-thalassemia, cystic fibrosis, hemophilia and neurodegenerative diseases, and mutations in genes involved in cancer.

A thorough list of the mutations in these genes is given on the following Internet site: ftp://ncbi.nlm.nih.gov/repository/OMIM/morbidmap.

The method and the kit according to the invention are also of use for studying the polymorphism of genes or any genetic region, and for detecting and/or identifying genetically modified organisms (GMOs).

### LEGENDS

FIG. 1A: solid-phase PCR amplification

Amplification of the genetic region of interest.

FIG. 1B: denaturation

The DNA double strand is denatured, and then the washes make it possible to conserve only the anchored strand.

FIG. 1C: incorporation of a modified nucleotide

An enzyme reaction catalyzes the elongation of a probe, resulting in the insertion of a modified nucleotide, for example an αS-phosphothioate deoxynucleotide, at the 3′ end of the probe. Two reactions in the two tubes A and B are necessary for each sample, with the incorporation of a single base to detect the normal (tube B) and/or mutant (tube A) genotypes.

FIG. 1D: detection

The elongated probe is protected against the degradation action of the exonuclease and a blue coloration develops during the incubation with the substrate. The nonelongated probe is eliminated, no coloration is obtained. For example, the detection can be carried out with an FITC-labeled probe and an anti-FITC labeled antibody.

FIG. 1E: very easy visual interpretation of the results

The results for 4 series of tubes A and B (for 4 individuals or for 4 different mutations in the same individual) are given. The positive results are blue (dark gray in the text) and the negatives are colorless. For each individual, whether mutated or normal, a coloration is obtained. In the heterozygous tests, a coloration is obtained in the two wells.

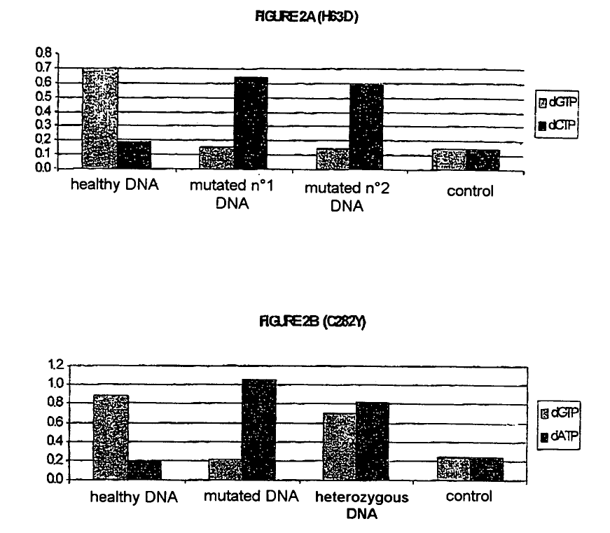

FIGS. 2A and 2B: detection of the mutations C282Y and H63D responsible for hemochromatosis.

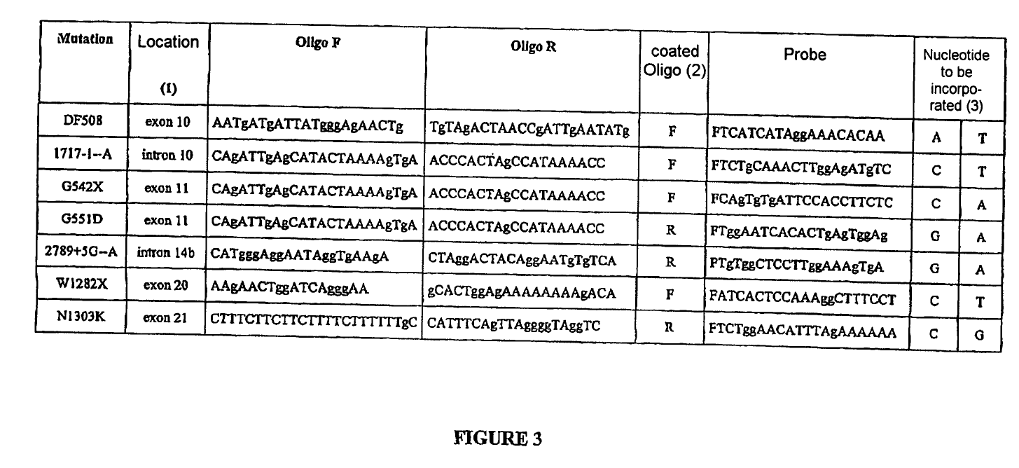

FIG. 3: primers used to detect the mutations responsible for cystic fibrosis

F=forward

R=reverse

(1) corresponds to the exon of the CFTR gene in which the mutation is found

(2) corresponds to the attached oligonucleotide=primer B

(3) corresponds to the modified nucleotide which will be incorporated during the extension: A=αS-dATP, T=αS-dTTP, C=αS-dCTP, G=αS-dGTP

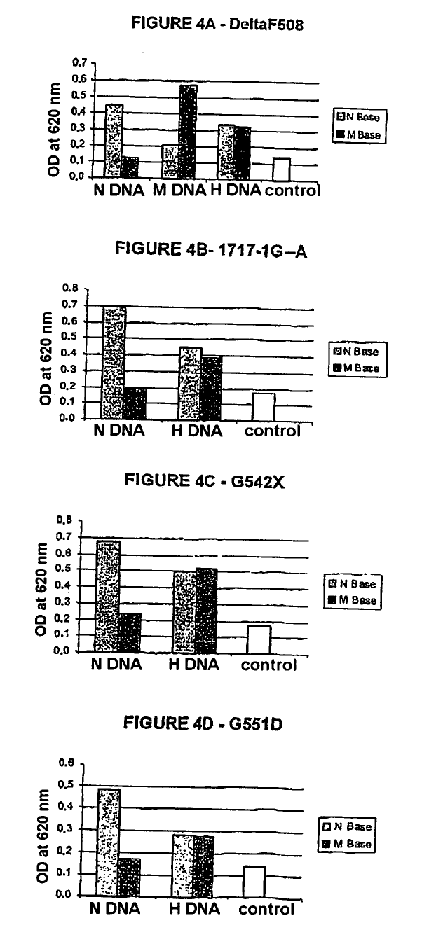

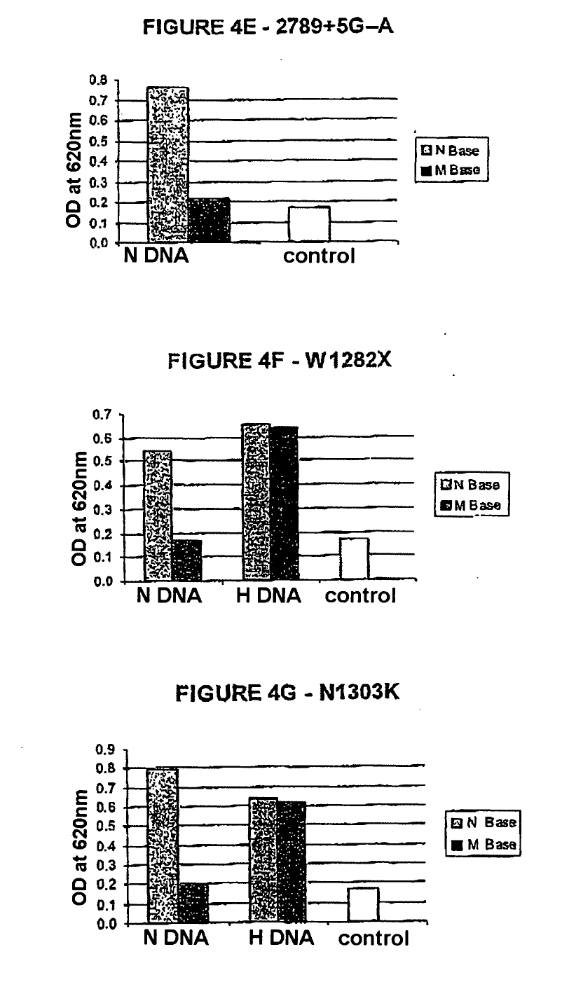

FIGS. 4A, 4B, 4C, 4D, 4E, 4F and 4G: detection of 7 mutations responsible for cystic fibrosis

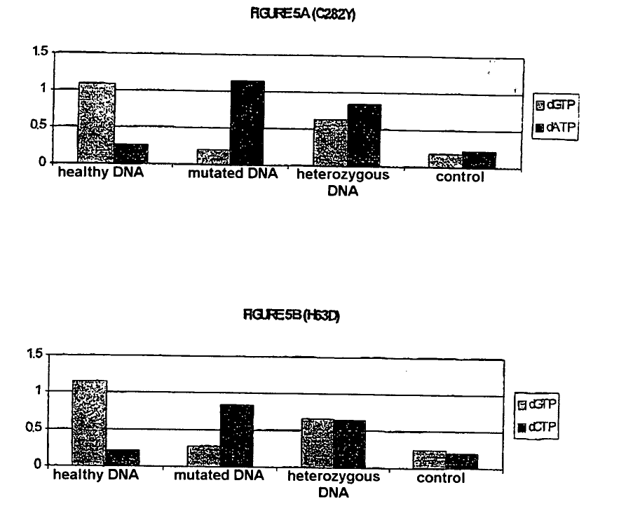

FIGS. 5A and 5B: detection of the mutations C282Y and H63D responsible for hemochromatosis (preferred method according to the invention in which the degradation and revelation steps are coupled).

### EXAMPLE 1

Protocol for Carrying Out the Method According to the Invention

The method according to the present invention makes it possible to determine the genotype of a sample, this sample being preamplified using a primer attached to the wall of the tube. The tubes used are Nucleolink strips (NUNC), and the attachment of the primer is carried out according to the supplier's indications.

1. PCR Amplification of the Region of Interest

Introduced into a tube containing the preattached primer B are the various components in accordance with the various suppliers and allowing amplification of the region to be analyzed by asymmetric PCR: PCR buffers, dNTP, primer A at a final concentration of 1 μM, primer B at a final concentration of 0.12 μM, Taq polymerase and DNA to be tested.

Comment: the amplification products obtained in liquid phase can be discarded or kept in order to be analyzed on a 1.5% agarose gel stained with ethidium bromide.

2. Dehybridization

the wells are washed with distilled water;

25 μl of sodium hydroxide (NaOH) (0.4 M) are placed in each well and the plate is left to stand for 5 minutes at ambient temperature;

the sodium hydroxide is removed and washing is carried out with distilled water.

3. Hybridization and Elongation

25 μl of hybridization solution N (normal) or M (mutant) are distributed into their respective tube; these two solutions are composed of 0.05 μM sodium hydroxide, 0.4 U Bst DNA polymerase (Biolabs), 1×Bst buffer, solution (2.5×SSC—0.25% blocking reagent (Amersham), and differ only by the presence of the 10 μM modified dNTP (dNTP*);

incubation is carried out for 15 minutes at 50° C.

4. Washes

The wells are emptied and washed with a 2.5% NaCl solution.

5. Enzyme Digestion

25 μl of solution containing 0.01 U of exonuclease III in its buffer (final concentrations: 50 mM Tris, 1 mM DTT, 10 mM MgCl₂) are deposited per well;

incubation is carried out for 10 minutes at 37° C.

6. Washes

The wells are emptied and washed with a 1×PBS washing solution containing 0.1% Tween.

7. Revelation

25 μl of anti-FITC-POD antibody (Boehringer) diluted to 1/2 000 in PBS-3% BSA are added to each well and left at ambient temperature for 10 minutes;

the well is emptied and washed with a 1×PBS solution containing 0.1% Tween;

25 μl of TMB (tetramethylbenzidine—Sigma) substrate are deposited per well;

incubation is carried out for 15 minutes at ambient temperature;

it is possible to stop the reaction with 25 μl of H₂SO₄.

8. Results

The reading may then be carried out with the naked eye; a blue coloration appears in the positive wells. It is also possible to read the optical density at 655 nm using a microplate reader, or at 450 nm if the reaction has been stopped.

9. Interpretation of the Results

Determination of the genotype of a given sample can be carried out:

by reading with the naked eye:

a blue coloration appears in the positive wells and the negative wells remain colorless. The four possible coloration profiles are described below:

| | | | |
|---|---|---|---|
| [symbol] | [symbol] | Case 1 | Normal |
| [symbol] | [symbol] | Case 2 | Mutant |
| [symbol] | [symbol] | Case 3 | Heterozygote |
| [symbol] | [symbol] | Case 4 | Negative control |

by reading the optical density at 655 nm (or 450 nm if the reaction is stopped).

The ratio R=[OD(N)/OD(M)] is determined.

If R>2 then the genotype is Normal.

If 0.5<R<2 then the genotype is Heterozygot.

If R<0.5 then the genotype is Mutant.

### EXAMPLE 2

Diagnosis of Hemochromatosis, Detection of the Mutations C282Y and H63D Responsible for Hemochromatosis

1. PCR Amplification of the Region of Interest

The following are introduced into a tube containing the attached primer B:

| | Final concentration | |
|---|---|---|
| 10X PCR buffer | 1X | |
| 10 mg/ml BSA | 1 | mg/ml |
| 50 mM MgCl₂ | 3 | mM |
| 10 mM dNTP | 0.20 | mM |
| 25 pmol/μl primer A | 0.5 | μM |
| 3 pmol/μl primer B | 0.06 | μM |
| 5 U/μl Taq polymerase | 0.02 | U/μl |
| sterile water qs 25 μl | 11.9 | μl |
| DNA to be tested | 1 | ng/μl |

2. Dehybridization

3. Hybridization and Elongation

25 μl of hybridization solution N (normal) or M (mutant) are distributed in their respective tube, the solutions different by αthiodGTP (normal base for C282Y and H63D) in the case of the normal solution; αthiodATP (mutant base for C282Y) in the case of the C282Y mutant solution or αthiodCTP (mutant base for H63D) in the case of the H63D mutant solution.

Detection of the Mutation C282Y:

Primers Used:

| | | |
|---|---|---|
| Primer A | 5′CATGAAGTGGCTGAAGGATAA3′ | (SEQ ID N°1) |
| Primer B | 5′GCACTCCTCTCAACCCCCA3′ | (SEQ ID N°2) |
| FITC-labeled probe | 5′FITC-GGAAGAGCAGAGATATACGT3′ | (SEQ ID N°3) |

Normal case: incorporation of an αS-dGTP, region of the SNP C282Y and its complementary region, included in the region amplified by the primers SEQ ID No. 1 and SEQ ID No. 2, the probe SEQ ID No. 3 being underlined, the polymorphic base being in bold.

| | |
|---|---|
| GGGAAGAGCAGAGATATACGTGATGAGAGT | (SEQ ID N°4) |
| CCCTTCTCGTCTCTATATGCACTACTCTCA | (SEQ ID N°5) |

Mutant case: incorporation of an αS-dATP, region of the SNP C282Y and its complementary region, included in the region amplified by the primers SEQ ID No. 1 and SEQ ID No. 2, the probe SEQ ID No. 3 being underlined, the polymorphic base being in bold.

| | |
|---|---|
| GGGAAGAGCAGAGATATACGTA | (SEQ ID N°7) |
| CCCTTCTCGTCTCTATATGCATTACTCTCA | |

Detection of the Mutation H63D:

Primers Used:

| | |
|---|---|
| Primer A | |
| 5′ACATCTGGCTTGAAATTCTACT3′ | (SEQ ID N°8) |
| Primer B | |
| 5′TCTCCAGGTTCACACTCTCT3′ | (SEQ ID N°9) |
| FITC-Iabeled probe | |
| 5′FITC-CCACACGGCGACTCTCAT3! | (SEQ ID N°10) |

Normal case: incorporation of an αS-dGTP, region of the SNP H63D and its complementary region, included in the region amplified by the primers SEQ ID No. 8 and SEQ ID No. 9, the probe SEQ ID No. 10 being underlined, the polymorphic base being in bold.

| | |
|---|---|
| TATGATCATGAGAGTCGCCGTGTGGAGCCC | (SEQ ID N°11) |
| ATACTACTACTCTCAGCGGCACACCTCGGG | (SEQ ID N°12) |

Mutant case: incorporation of an αS-dCTP, region of the SNP H63D and its complementary region, included in the region amplified by the primers SEQ ID No. 8 and SEQ ID No. 9, the probe SEQ ID No. 10 being underlined, the polymorphic base being in bold.

| | |
|---|---|
| TATGATGATGAGAGTCGCCGTGTGGAGCCC | (SEQ ID N°13) |
| ATACTACTACTCTCAGCGGCACACCTCGGG | (SEQ ID N°14) |

The results are given in FIG. 2.

### EXAMPLE 3

Diagnosis of Cystic Fibrosis, Detection of Seven Mutations Responsible for Cystic Fibrosis

| | | | | | |
|---|---|---|---|---|---|
| | | Fre- | | | |
| | | quen- | | | |
| | | cies | | | |
| Mutation | Location | (%)* | Oligo F | Oligo R | Probe |

| | | | | | |
|---|---|---|---|---|---|
| DF508 | exon 10 | 66.11 | SEQ ID 15 | SEQ ID 16 | SEQ ID 17 |
| 1717− | intron 10 | 2.6 | SEQ ID 18 | SEQ ID 19 | SEQ ID 20 |
| 1G−A | | | | | |
| G542X | exon 11 | 2.11 | SEQ ID 21 | SEQ ID 22 | SEQ ID 23 |
| G551D | exon 11 | 1.69 | SEQ ID 24 | SEQ ID 25 | SEQ ID 26 |
| 2789+ | intron 14b | 1.15 | SEQ ID 27 | SEQ ID 28 | SEQ ID 29 |
| 5G−A | | | | | |
| W1282X | exon 20 | 0.88 | SEQ ID 30 | SEQ ID 31 | SEQ ID 32 |
| N1303K | exon 21 | 0.76 | SEQ ID 33 | SEQ ID 34 | SEQ ID 35 |

Frequencies (%)*: frequencies calculated according to Cystic Fibrosis Mutation Data Base February 2000 (http://www.genet.cikkids.on.ca/cftr/newfreq/All.html).

Primers and Probes Used (See FIG. 3)

Comment: The protocol used is identical to the general description, the use of Nucleolink strips (NUNC) makes it possible to test, on a 96-well format, 7 mutations in 6 individuals. The results are given in FIG. 4.

### EXAMPLE 4

Improvement of the Protocol, Coupling of the Exonuclease Digestion and Revelation Steps

Steps 1 to 4 are identical to those of example 1, only the following steps are modified such that the digestion with exonuclease III and the revelation with the antibody are carried out in a single step.

25 μl of solution containing 0.01 U of exonuclease III (New England Biolabs) in its buffer, 2.5 μl of anti-FITC-POD antibody (Boehringer Mannheim) diluted to 1/200 in PBS-3% BSA, and 0.25 μl of Tween 20 are deposited per well;

incubation is carried out for 10 minutes at 37° C.;

3 washes are carried out with the 1×PBS washing solution containing 0.1% Tween;

25 μl of TMB are deposited and incubation is carried out for 15 minutes at 37° C.

The results given in FIG. 5 are interpreted as indicated in example 1.

### EXAMPLE 5

Improvement of the Protocol, Using a Streptavidin/Biotin System (Probe Biotinylated and Streptavidin Conjugated)

Steps 1 and 2 are identical to those of example 1, only the following steps are modified such that the final revelation is carried out by virtue of streptavidin, the probe having been coupled to a biotin beforehand.

3. Hybridization and Elongation

25 μl of hybridization solution N (normal) or M (mutant) are distributed in their respective tube; these two solutions are composed of: 0.05 μM biotinylated probe, 0.4 U Bst polymerase (New England Biolabs), 10×Bst buffer: 2.5 μl, solution (2.5 SSC, 0.25% blocking reagent (Amersham)), and differ only by the presence of the 10 μM modified dNTP.

Incubation is carried out for 15 minutes at 50° C.

4. Washes

The wells are emptied and washed with a solution containing 2.5% NaCl.

5. Enzyme Digestion

25 μl of solution containing 0.01 U of exonuclease III (New England Biolabs) in its buffer are deposited per well;

incubation is carried out for 10 minutes at 37° C.

6. Washes

The wells are emptied and 3 washes are carried out with a 1×PBS washing solution containing 0.1% Tween.

7. Revelation

25 μl of 1 mg/ml streptavidin diluted to 1/2000 in PBS are added;

the mixture is left to incubate for 10 minutes at ambient temperature.

8. 3 washes are carried out with the 1×PBS washing solution containing 0.1% Tween. 9. 25 μl of TMB are deposited and incubation is carried out for 15 minutes at 37° C.

10. Results and interpretation of the results are as indicated in example 1.

1. A method for detecting a mutation occurring at position n in a target nucleic acid, characterized in that it comprises the following steps:
a) amplification on at least two different solid supports A and B of the region of interest comprising said mutation using at least one primer bound in the 5′ position to the supports,
b) dehybridization of the DNA strands and removal of the strands of the suspension by washing,
c) hybridization of a probe with the DNA strands bound to the solid supports A and B, the 3′ end of said probe hybridizing up to at most nucleotide n-1 of said strands,
d) reaction consisting of elongation of the probe hybridized in step c), by incorporation, in the 5′-3′ direction, of nucleotides complementary to said DNA strands using a reaction mixture comprising a DNA polymerase and a nucleotide derivative resistant to degradation by an exonuclease (dNTP*), the reaction mixture used for the support A comprising a dNTP* complementary to said mutation and the reaction mixture used for the support B comprising a dNTP* complementary to the normal base corresponding to said mutation,
e) degradation with said exonuclease such that only the probes elongated in step d) are not degraded, washing,
f) direct or indirect revelation of the nondegraded probe, said revelation being positive either when the DNA strand on the support A contains the mutation, or when the DNA strand on the support B contains the normal base corresponding to said mutation.

2. The method as claimed in claim 1, characterized in that the probe is labeled with molecules selected from enzymes, in particular enzymes capable of acting on a chromogenic, fluorigenic or luminescent substrate (in particular a peroxidase or an alkaline phosphatase), chemical chromophore compounds, chromogenic, fluorigenic or luminescent compounds, nucleotide based analogs, and ligands such as biotin, said molecules allowing the direct or indirect appearance of a coloration when the nondegraded probe is present on the support, said coloration possibly being detected preferably by optical measurement or simple observation.

3. The method as claimed in either of claims 1 and 2, characterized in that, in step f), the presence or absence of the mutations is detected either by optical reading, or by simple observation of a coloration on the solid support.

4. The method as claimed in one of claims 1 to 3, characterized in that the supports A and B are tubes to which covalent attachment of nucleic acids can be carried out, preferably tubes made of a plastic material such as polystyrene or polycarbonate, in particular tubes of the Nucleolink™ type.

5. The method as claimed in one of the preceding claims, characterized in that, in step d), an αS-phosphothioate deoxynucleotide is used, preferably αS-dATP, αS-dTTP, αS-dCTP, αS-dGTP, αS-dUTP or αS-dITP.

6. The method as claimed in one of the preceding claims, characterized in that, in step e), exonuclease III is used.

7. The method as claimed in one of the preceding claims, characterized in that two tubes are used, one being intended for detection of the mutated base (tube A) and the other being intended for detection of the normal base corresponding to said mutated base (tube B).

8. The method as claimed in one of the preceding claims, characterized in that several series of tubes A and B are used, in particular in a 96-well plate.

9. The method as claimed in one of the preceding claims, characterized in that a tube C is also used, as a negative control.

10. The method as claimed in one of the preceding claims, characterized in that, steps e) and f) are carried out simultaneously in the same reaction mixture comprising said exonuclease and the means necessary to reveal the coloration.

11. A device or kit for carrying out the method as claimed in one of the preceding claims, characterized in that it comprises:
tubes A and B in which at least one primer for amplifying the region comprising the specific mutation is bound, in the 5′ position, via a covalent bond,
reaction mixtures A and B, each comprising a different αS-phosphothioate deoxynucleotide selected from αS-dATP, αS-dTTP, αS-dCTP, αS-dGTP, αS-dUTP and αS-dITP, the reaction mixture used for the support A comprising an αS-phosphothioate deoxynucleotide complementary to said mutation and the reaction mixture used for the support B comprising an αS-phosphothioate deoxynucleotide complementary to the normal base corresponding to said mutation.

12. The kit as claimed in claim 11, characterized in that it also comprises at least one element selected from an exonuclease, in particular exonuclease III, a reagent for revealing the coloration, a DNA polymerase, and various buffers or solutions required to carry out the method.

13. The kit as claimed in either of claims 11 and 12, characterized in that it comprises a series of tubes A and B, each series making it possible to detect a given mutation.

14. The kit as claimed in one of claims 11 to 13, characterized in that it also comprises tubes C as negative controls.

15. The kit as claimed in one of claims 11 to 14, characterized in that said tubes are tubes made of a plastic material such as polystyrene or polycarbonate, in particular tubes of the Nucleolink™ type.

16. The use of the method as claimed in one of claims 1 to 10 and of the kit as claimed in one of claims 11 to 15, for detecting mutations of genes involved in diseases, in particular in hereditary genetic diseases, in particular hemochromatosis, sickle cell anemia, β-thalassemia and α-thalassemia, cystic fibrosis, hemophilia and neurodegenerative diseases, and mutations in genes involved in cancer.

17. The use of the method as claimed in one of claims 1 to 10 and of the kit as claimed in one of claims 11 to 16, for studying the polymorphism of genes or any genetic region.

18. The use of the method as claimed in one of claims 1 to 10 and of the kit as claimed in one of claims 11 to 16, for detecting and/or identifying genetically modified organisms (GMOs).


SEQUENCE LISTING (35 sequences)

SEQ ID NO: 1 - DNA, 21 bp, Homo sapiens
  Detection of C282Y mutation in the hemochromatosis gene.
catgaagtggctgaaggataa

SEQ ID NO: 2 - DNA, 19 bp, Homo sapiens
  Detection of C282Y mutation in the hemochromatosis gene.
gcactcctctcaaccccca

SEQ ID NO: 3 - DNA, 20 bp, Homo sapiens
  modified_base (1): Probe marked FITC in 5′.
ggaagagcagagatatacgt

SEQ ID NO: 4 - DNA, 30 bp, Homo sapiens
  Detection of C282Y mutation in the hemochromatosis gene.
gggaagagcagagatatacgtgatgagagt

SEQ ID NO: 5 - DNA, 30 bp, Homo sapiens
  Detection of C282Y mutation in the hemochromatosis gene.
cccttctcgtctctatatgcactactctca

SEQ ID NO: 6 - DNA, 30 bp, Homo sapiens
  Detection of C282Y mutation in the hemochromatosis gene.
gggaagagcagagatatacgtaatgagagt

SEQ ID NO: 7 - DNA, 30 bp, Homo sapiens
  Detection of C282Y mutation in the hemochromatosis gene.
cccttctcgtctctatatgcattactctca

SEQ ID NO: 8 - DNA, 22 bp, Homo sapiens
  Detection of H63D mutation in the hemochromatosis gene.
acatctggcttgaaattctact

SEQ ID NO: 9 - DNA, 20 bp, Homo sapiens
  Detection of H63D mutation in the hemochromatosis gene.
tctccaggttcacactctct

SEQ ID NO: 10 - DNA, 18 bp, Homo sapiens
  modified_base (1): Probe marked FITC in 5′.
ccacacggcgactctcat

SEQ ID NO: 11 - DNA, 30 bp, Homo sapiens
  Detection of H63D mutation in the hemochromatosis gene.
tatgatcatgagagtcgccgtgtggagccc

SEQ ID NO: 12 - DNA, 30 bp, Homo sapiens
  Detection of H63D mutation in the hemochromatosis gene.
atactagtactctcagcggcacacctcggg

SEQ ID NO: 13 - DNA, 30 bp, Homo sapiens
  Detection of H63D mutation in the hemochromatosis gene.
tatgatgatgagagtcgccgtgtggagccc

SEQ ID NO: 14 - DNA, 30 bp, Homo sapiens
  Detection of H63D mutation in the hemochromatosis gene.
atactactactctcagcggcacacctcggg

SEQ ID NO: 15 - DNA, 22 bp, Homo sapiens
  Primer for the detection of the mutations associated with mucoviscidosis.
aatgatgattatgggagaactg

SEQ ID NO: 16 - DNA, 23 bp, Homo sapiens
  Primer for the detection of the mutations associated with mucoviscidosis.
tgtagactaaccgattgaatatg

SEQ ID NO: 17 - DNA, 18 bp, Homo sapiens
  modified_base (1): Probe marked FITC in 5′.
tcatcataggaaacacaa

SEQ ID NO: 18 - DNA, 23 bp, Homo sapiens
  Primer for the detection of the mutations associated with mucoviscidosis.
cagattgagcatactaaaagtga

SEQ ID NO: 19 - DNA, 19 bp, Homo sapiens
  Primer for the detection of the mutations associated with mucoviscidosis.
acccactagccataaaacc

SEQ ID NO: 20 - DNA, 20 bp, Homo sapiens
  modified_base (1): Probe marked FITC in 5′.
tctgcaaacttggagatgtc

SEQ ID NO: 21 - DNA, 23 bp, Homo sapiens
  Primer for the detection of the mutations associated with mucoviscidosis.
cagattgagcatactaaaagtga

SEQ ID NO: 22 - DNA, 19 bp, Homo sapiens
  Primer for the detection of the mutations associated with mucoviscidosis.
acccactagccataaaacc

SEQ ID NO: 23 - DNA, 20 bp, Homo sapiens
  modified_base (1): Probe marked FITC in 5′.
cagtgtgattccaccttctc

SEQ ID NO: 24 - DNA, 23 bp, Homo sapiens
  Primer for the detection of the mutations associated with mucoviscidosis.
cagattgagcatactaaaagtga

SEQ ID NO: 25 - DNA, 19 bp, Homo sapiens
  Primer for the detection of the mutations associated with mucoviscidosis.
acccactagccataaaacc

SEQ ID NO: 26 - DNA, 20 bp, Homo sapiens
  modified_base (1): Probe marked FITC in 5′.
tggaatcacactgagtggag

SEQ ID NO: 27 - DNA, 21 bp, Homo sapiens
  Primer for the detection of the mutations associated with mucoviscidosis.
catgggaggaataggtgaaga

SEQ ID NO: 28 - DNA, 22 bp, Homo sapiens
  Primer for the detection of the mutations associated with mucoviscidosis.
ctaggactacaggaatgtgtca

SEQ ID NO: 29 - DNA, 20 bp, Homo sapiens
  modified_base (1): Probe marked FITC in 5′.
tgtggctccttggaaagtga

SEQ ID NO: 30 - DNA, 18 bp, Homo sapiens
  Primer for the detection of the mutations associated with mucoviscidosis.
aagaactggatcagggaa

SEQ ID NO: 31 - DNA, 21 bp, Homo sapiens
  Primer for the detection of the mutations associated with mucoviscidosis.
gcactggagaaaaaaaagaca

SEQ ID NO: 32 - DNA, 20 bp, Homo sapiens
  modified_base (1): Probe marked FITC in 5′.
atcactccaaaggctttcct

SEQ ID NO: 33 - DNA, 24 bp, Homo sapiens
  Primer for the detection of the mutations associate with mucoviscidosis.
ctttcttcttcttttcttttttgc

SEQ ID NO: 34 - DNA, 21 bp, Homo sapiens
  Primer for the detection of the mutations associated with mucoviscidosis.
catttcagttaggggtaggtc

SEQ ID NO: 35 - DNA, 20 bp, Homo sapiens
  modified_base (1): Probe marked FITC in 5′.
tctggaacatttagaaaaaa